(19) United States
(12) Patent Application Publication
(10) Pub. No.: US 2015/0037255 A1
(43) Pub. Date: Feb. 5, 2015

(54) PHTHALOCYANINE DYE-CONTAINING CONTRAST AGENT FOR PHOTOACOUSTIC IMAGING

(71) Applicant: CANON KABUSHIKI KAISHA, Tokyo (JP)

(72) Inventors: Tatsuki Fukui, Yokohama-shi (JP); Mayuko Kishi, Machida-shi (JP); Fumiko Tomatsu, Yokohama-shi (JP); Daisuke Sasaguri, Yokohama-shi (JP); Satoshi Yuasa, Yokohama-shi (JP)

(21) Appl. No.: 14/333,704

(22) Filed: Jul. 17, 2014

(30) Foreign Application Priority Data
Jul. 30, 2013 (JP) 2013-158052

(51) Int. Cl. A61K 49/22

(52) U.S. Cl. CPC A61K 49/22
USPC 424/9.1

(57) ABSTRACT

To provide a particle promoting hydrophobic metal phthalocyanine aggregation, having absorption in a wavelength region suitable for a photoacoustic imaging method and having a high molar absorbance coefficient per particle by increasing the weight percentage of the dye in the particle. The particle of the present invention is a particle having a hydrophobic metal phthalocyanine and a surfactant, wherein the weight percentage of the hydrophobic metal phthalocyanine is 6% or more.

### BACKGROUND OF THE INVENTION

1. Field of the Invention

The present invention relates to a contrast agent for photoacoustic imaging, having a hydrophobic metal phthalocyanine.

2. Description of the Related Art

Attention has been given in recent years to a fluorescent imaging method or a photoacoustic imaging method as an imaging method capable of noninvasive diagnosis.

The fluorescent imaging method is a method involving irradiating a fluorescent dye with light and detecting fluorescence emitted by the dye and is widely used. The photoacoustic imaging method is a method involving obtaining the imaging of an object to be measured by detecting the intensity and generation position of an acoustic wave resulting from volume expansion caused by heat released from a molecule as the object to be measured irradiated with light. The fluorescent imaging method or the photoacoustic imaging method can use a dye as a contrast agent for increasing the intensity of fluorescence or an acoustic wave from a site to be measured.

Photodynamic therapy uses zinc phthalocyanine (hereinafter sometimes abbreviated as ZnPc) as a dye known to absorb light as a photosensitive substance.

National Publication of International Patent Application No. H11-514986 (hereinafter referred to as Patent Literature 1) discloses a particle using ZnPc and a pharmaceutically acceptable polymer suitable for the formation of nanoparticles.

Journal of Controlled Release, 155 (3), pp. 400-408 (hereinafter referred to as Non-Patent Literature 1) discloses a particle using hydrophilized ZnPc and canola oil.

However, the ZnPc-containing particle disclosed in Patent Literature 1 has the dye dispersed therein because of being intended to be used for photodynamic therapy, and has the problem of having a low content of the dye and having a low molar absorbance coefficient per particle.

The ZnPc-containing particle disclosed in Non-Patent Literature 1 also has the dye dispersed therein because of being intended to be used for photodynamic therapy, and has no absorption in a wavelength region suitable for a photoacoustic imaging method.

### SUMMARY OF THE INVENTION

Accordingly, an object of the present invention is to provide a particle promoting hydrophobic metal phthalocyanine aggregation, having absorption in a wavelength region suitable for a photoacoustic imaging method and having a high molar absorbance coefficient per particle by increasing the weight percentage of the dye in the particle.

The present invention relates to a particle having a hydrophobic metal phthalocyanine and a surfactant, wherein the weight percentage of the hydrophobic metal phthalocyanine is 6% or more and the hydrophobic metal phthalocyanine is represented by general formula (1):

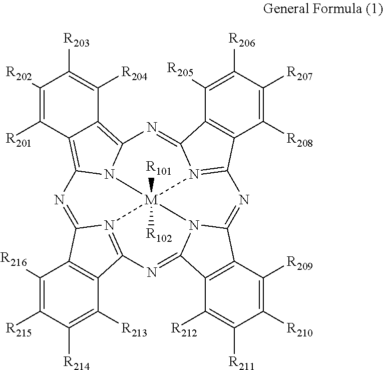

wherein R₂₀₁ to R₂₁₆ may be identical or different and each represent a hydrogen atom, a halogen atom, an acetoxy group, an amino group, a nitro group, a cyano group or an alkyl group having 1 to 18 carbons or an aromatic group, wherein the aromatic group is one unsubstituted or substituted with one or more functional groups selected from the group consisting of halogen atoms, an acetoxy group, an amino group, a nitro group, a cyano group, and alkyl groups each having 1 to 18 carbons;

M represents a Zn, Cu, Co or Si element;

R₁₀₁ and R₁₀₂ may be identical or different, may be absent depending on the element of M, or are each represented by the structure shown below:

—OH, —OR₁₁, —OCOR₁₂, —OSi(—R₁₃)(—R₁₄)(—R₁₅), a halogen atom, an acetoxy group, an amino group, a nitro group, a cyano group or an alkyl group having 1 to 18 carbons or an aromatic group, wherein the aromatic group is one unsubstituted or substituted with one or more functional groups selected from the group consisting of halogen atoms, an acetoxy group, an amino group, a nitro group, a cyano group, and alkyl groups each having 1 to 18 carbons,

wherein R₁₁ to R₁₅ are each selected from the group consisting of halogen atoms, an acetoxy group, an amino group, a nitro group, a cyano group, and alkyl groups each having 1 to 18 carbons, wherein R₁₃, R₁₄ and R₁₅ may be identical or different.

Further features of the present invention will become apparent from the following description of exemplary embodiments with reference to the attached drawings.

### BRIEF DESCRIPTION OF THE DRAWINGS

FIG. 1 is a diagram for illustrating a particle according to embodiment 1 of the present invention.

FIG. 2 is a diagram for illustrating a particle according to embodiment 2 of the present invention.

FIG. 3 is a graph showing the relation between weight percentage of dye in particle and photoacoustic signal per particle in terms of 100 nm obtained in experimental example D5.

### DESCRIPTION OF THE EMBODIMENTS

Preferred embodiments of the present invention will now be described in detail in accordance with the accompanying drawings.

Embodiments of the present invention will be described below. However, the present invention is not intended to be limited thereto.

(Construction of Embodiment)

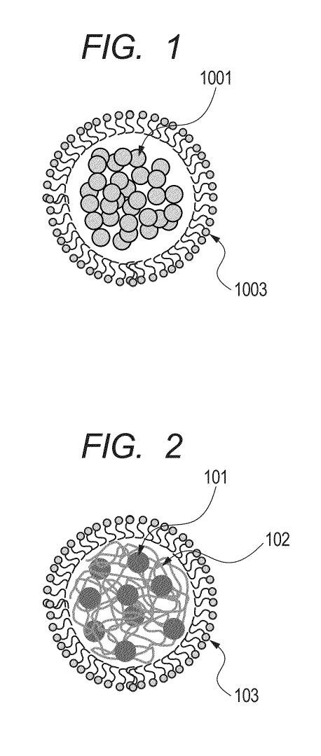

As shown in FIG. 1, the particle according to the present embodiment is a particle having a hydrophobic metal phthalocyanine 1001, and a surfactant 1003 in the particle surface, wherein the hydrophobic dye is represented by the general formula (1). As shown in FIG. 2, the particle according to the present embodiment is a particle having a hydrophobic metal phthalocyanine 101, a surfactant 103 in the particle surface, and optionally a matrix material 102 for including the hydrophobic dye. The particle is characterized by having a weight percentage of a hydrophobic metal phthalocyanine in the particle of 6% or more.

The higher weight percentage of the hydrophobic metal phthalocyanine facilitates the aggregation of hydrophobic metal phthalocyanine molecules, red-shifts the absorption band and results in the particle having absorption in a wavelength region suitable for a photoacoustic imaging method.

The hydrophobic metal phthalocyanine according to each of the present embodiments is less easily leaked to the outside of the particle in an aqueous solution such as serum because of having a highly hydrophobic structure as described above and having no hydrophilic functional groups. Hydrophobic metal phthalocyanine molecules are dye molecules; these molecules hydrophobically act on each other and less easily leaked from the particle.

As disclosed in Drug Delivery System 25(5), pp. 448-455, 2010 (hereinafter referred to as Non-Patent Literature 2) and Abstracts of the Annual Meeting of the Tokai Branch of the Japanese Society of Hospital Pharmacists, 19th, 116 (hereinafter referred to as Non-Patent Literature 3), in the development of a material used for photodynamic therapy, the aggregation of dye molecules is the problem of impairing a therapeutic effect, which is contrary to the object of the present invention.

(Hydrophobic Metal Phthalocyanine)

The hydrophobic metal phthalocyanine is a dye. According to the present invention, the dye is defined as a compound capable of absorbing light of a wavelength included in the range of 600 nm to 1,300 nm.

According to the present embodiments, the hydrophobic dye is defined as a dye whose Rf value is 0 to 0.50 (both inclusive) as calculated by a thin-layer liquid chromatography (hereinafter sometimes abbreviated as TLC) method, described later in Examples.

According to the present embodiments, the hydrophobic metal phthalocyanine structure is represented by general formula (1) below:

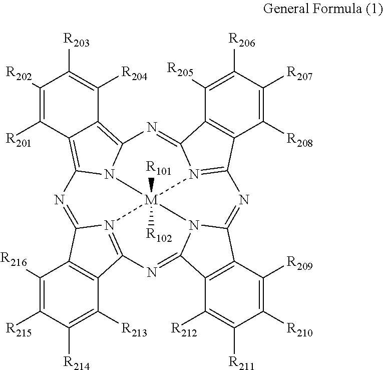

wherein R₂₀₁ to R₂₁₆ may be identical or different and each represent a hydrogen atom, a halogen atom, an acetoxy group, an amino group, a nitro group, a cyano group or an alkyl group having 1 to 18 carbons or an aromatic group, wherein the aromatic group is one unsubstituted or substituted with one or more functional groups selected from the group consisting of halogen atoms, an acetoxy group, an amino group, a nitro group, a cyano group, and alkyl groups each having 1 to 18 carbons;

M represents a Zn, Cu, Co or Si element;

R₁₀₁ and R₁₀₂ may be identical or different, may be absent depending on the element of M, or are each represented by the structure shown below:

—OH, —OR₁₁, —OCOR₁₂, —OSi(—R₁₃)(—R₁₄)(—R₁₅), a halogen atom, an acetoxy group, an amino group, a nitro group, a cyano group or an alkyl group having 1 to 18 carbons or an aromatic group, wherein the aromatic group is one unsubstituted or substituted with one or more functional groups selected from the group consisting of halogen atoms, an acetoxy group, an amino group, a nitro group, a cyano group, and alkyl groups each having 1 to 18 carbons,

wherein R₁₁ to R₁₅ are each selected from the group consisting of halogen atoms, an acetoxy group, an amino group, a nitro group, a cyano group, and alkyl groups each having 1 to 18 carbons, wherein R₁₃, R₁₄ and R₁₅ may be identical or different.

The hydrophobic metal phthalocyanine according to the present embodiments can absorb light of a particular wavelength because of having conjugated double bonds and can be used for photoacoustic imaging and fluorescent imaging.

The hydrophobic metal phthalocyanine according to the present embodiments can have a molar absorbance coefficient of 10⁶ M⁻¹ cm⁻¹ or more at least one wavelength selected from the group consisting of the range of 600 nm to 1,300 nm.

In addition, the weight percentage of the hydrophobic dye in the particle can be 6% or more.

Examples of the hydrophobic metal phthalocyanine dye can include zinc 2,9,16,23-tetra-tert-butyl-29H,31H-phthalocyanine, copper (II) 2,9,16,23-tetra-tert-butyl-29H,31H-phthalocyanine, cobalt 2,9,16,23-tetra-tert-butyl-29H,31H-phthalocyanine, and tert-butyl silicon-[bis trimethylsiloxy]-phthalocyanine.

Zinc 2,9,16,23-tetra-tert-butyl-29H,31H-phthalocyanine is represented by the following formula.

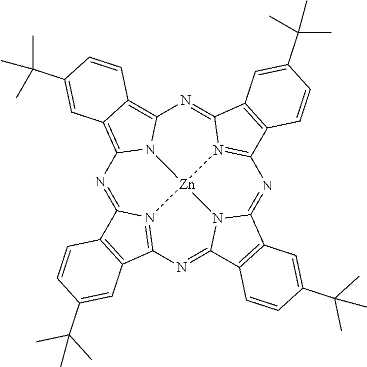

Copper (II) 2,9,16,23-tetra-tert-butyl-29H,31H-phthalocyanine is represented by the following formula.

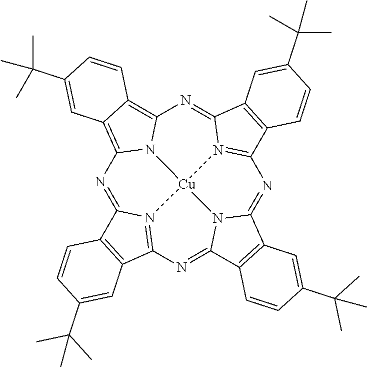

Cobalt 2,9,16,23-tetra-tert-butyl-29H,31H-phthalocyanine is represented by the following formula.

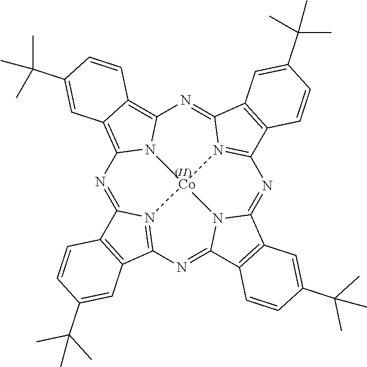

(Surfactant)

The particle according to each of the present embodiments has a surfactant. The surfactant according to the present embodiments is not particularly limited and may be any surfactant provided that the particle can be formed. Examples thereof which can be used include non-ionic surfactants, anionic surfactants, cationic surfactants, macromolecular surfactants and phospholipids. These surfactants may be used alone or in a combination of 2 or more thereof.

Examples of the non-ionic surfactants can include polyoxyethylene sorbitan fatty acid esters such as Tween (R) 20, Tween (R) 40, Tween (R) 60, Tween (R) 80 and Tween (R) 85, Brij (R) 35, Brij (R) 58, Brij (R) 76, Brij (R) 98, Triton (R) X-100, Triton (R) X-114, Triton (R) X-305, Triton (R) N-101, Nonidet (R) P-40, IGEPAL (R) CO530, IGEPAL (R) CO630, IGEPAL (R) CO720 and IGEPAL (R) CO730. Among these non-ionic surfactants, Tween 20 or Tween 80 can be used.

Examples of the anionic surfactants can include sodium dodecyl sulfate, dodecylbenzenesulfonates, decylbenzenesulfonates, undecylbenzenesulfonates, tridecylbenzenesulfonates, nonylbenzenesulfonates and sodium, potassium and ammonium salts thereof.

Examples of the cationic surfactants can include cethyltrimethylammonium bromide, hexadecylpyridinium chloride, dodecyltrimethylammonium chloride and hexadecyltrimethylammonium chloride.

Examples of the macromolecular surfactants can include polyvinyl alcohol and polyoxyethylene polyoxypropylene glycol. Examples of commercially available polyoxyethylene polyoxypropylene glycol can include Pluronic F68 (from BASF Co., Ltd.) and Pluronic F127 (from BASF Co., Ltd.).

Examples of the phospholipids can include phosphatidyl phospholipids each having any functional group of an amino group, an NHS group, a maleimide group and a methoxy group and a PEG chain.

Examples of the phosphatidyl phospholipids can include 3-(N-succinimidyloxyglutaryl)aminopropyl, polyethyleneglycol-carbamyl distearoylphosphatidyl-ethanolamine (DSPE-PEG-NHS), N-(3-maleimide-1-oxopropyl)aminopropyl polyethyleneglycol-carbamyl distearoylphosphatidyl-ethanolamine (DSPE-PEG-MAL), N-(aminopropyl polyethyleneglycol)-carbamyl distearoylphosphatidyl-ethanolamine (DSPE-PEG-NH₂), N-(carbonyl-methoxypolyethyleneglycol 2000)-1,2-distearoyl-sn-glycero-3-phosphoethanolamine, sodium salt (SUNBRIGHT DSPE-020CN), and N-(carbonyl-methoxypolyethyleneglycol 5000)-1,2-distearoyl-sn-glycero-3-phosphoethanolamine, sodium salt (SUNBRIGHT DSPE-050CN).

(Matrix Material)

The matrix material may be any material capable of including the hydrophobic metal phthalocyanine dye; however, the material can be a hydrophobic polymer because the use of the polymer increases hydrophobic interaction with the hydrophobic dye and can efficiently prevent the leakage of the dye.

(Hydrophobic Polymer)

Examples of the hydrophobic polymer according to the present embodiment include a homopolymer of monomers with hydroxycarboxylic acid, having 6 carbons or less, and a copolymer of 2 or more types of the monomers.

When the contrast agent according to each of the present embodiments is administered in vivo, a polymer of monomers with hydroxycarboxylic acid, having 6 carbons or less can be used as the hydrophobic polymer to avoid the in vivo remaining of the contrast agent over a long period of time. This is because the polymer of monomers with hydroxycarboxylic acid, having 6 carbons or less has ester bonds capable of being cleaved by enzyme in vivo. The polymer whose ester bonds are cleaved is less likely to remain in vivo because of being easily metabolized.

Examples of the polymer of monomers with hydroxycarboxylic acid, having 6 carbons or less include polylactic acid (PLA), polyglycolic acid (PGA) and poly(lactide-co-glycolide) copolymers (PLGA).

The hydrophobic polymer may have a hydrophilic portion. Examples of such a polymer include polymethyl methacrylate, polyethyl methacrylate, polybutyl methacrylate and polyisobutyl methacrylate.

The hydrophobic polymer preferably has a weight average molecular weight of 2,000 to 1,000,000, more preferably 10,000 to 600,000.

The matrix material in the particle according to the present embodiment can be particularly PLGA. PLGA are expected to be easily carried out of the body after imaging because of being susceptible to hydrolysis and be less easily accumulated in vivo. The composition ratio of lactic acid to glycolic acid in PLGA is not particularly limited and PLGA with any ratio can be used; however, PLGA having lactic acid to glycolic acid composition ratios of 25:75, 50:50 and 75:25 can be given as a preferable example. Lactic acid constituting PLGA can be used in any mixture among the D-form, L-form, racemic form and the like thereof.

(Method for Producing Particle)

A well-known method can be used as a method for producing the particle of the present invention; for example, a nanoemulsion method or a nanoprecipitation method can be used.

Examples of the solvent used for the present production method can include hydrocarbons such as hexane, cyclohexane and heptane, ketones such as acetone and methyl ethyl ketone, ethers such as diethyl ether and tetrahydrofuran, halogenated hydrocarbons such as dichloromethane, chloroform, carbon tetrachloride, dichloroethane and trichloroethane, aromatic hydrocarbons such as benzene and toluene, esters such as ethyl acetate and butyl acetate, aprotic polar solvents such as N,N-dimethylformamide and dimethyl sulfoxide and pyridine derivatives. These solvents may be used alone or in any mixture thereof.

The nanoemulsion method is a heretofore known method involving producing particles by preparing an emulsion by an emulsification technique. Examples thereof include stirring methods using mixers such as an intermittent shaking method, a propeller stirrer and a turbine stirrer, a colloid mill method, a homogenizer method and an ultrasonic irradiation method. These methods may be used alone or in a combination of a plurality thereof. The emulsion may be prepared by one-step emulsification or may be prepared by multi-step emulsification. However, the emulsification technique is not limited to the above techniques in the range allowing the achievement of the object of the present invention.

The nanoprecipitation method is a heretofore known method involving obtaining particles by mixing an organic solvent dispersion in a surfactant aqueous dispersion before stirring or a heretofore known method involving obtaining particles by mixing a surfactant aqueous dispersion in an organic solvent dispersion before stirring.

(Organic Solvent Dispersion in which Material Containing Hydrophobic Metal Phthalocyanine Dye is Dissolved)

In the nanoemulsion method, the weight ratio of the surfactant aqueous dispersion to the organic solvent dispersion used is not particularly limited provided that an oil-in-water (O/W) type emulsion can be formed. The weight ratio of the organic solvent dispersion to the aqueous dispersion can be in the range of 1:2 to 1:1,000.

In the nanoprecipitation method, the weight ratio of the surfactant aqueous dispersion to the organic solvent dispersion used is not particularly limited provided that particles can be recovered. The weight ratio of the organic solvent dispersion to the aqueous dispersion can be in the range of 1:1 to 1:1,000.

(Concentration of Material in Organic Solvent Dispersion in which Material Containing Hydrophobic Metal Phthalocyanine Dye is Dissolved)

The concentration of the hydrophobic metal phthalocyanine dye in the organic solvent dispersion is not particularly limited provided that the concentration is in the range allowing their dissolution. The concentration can be 0.0005 to 300 mg/ml.

The concentrations of the polymer and the hydrophobic metal phthalocyanine dye in the organic solvent dispersion are not particularly limited provided that the concentrations are in the ranges allowing their dissolution. The concentration of the polymer can be 0.3 to 100 mg/ml. The weight ratio of the hydrophobic metal phthalocyanine dye and the polymer in the organic solvent dispersion can be 100:1 to 1:1,000.

(Removal of Organic Solvent by Distillation from Particle Dispersion)

The removal by distillation can be carried out by any heretofore known method; however, examples thereof can include a method involving removal by heating and a method using a decompression apparatus such as an evaporator.

In the nanoemulsion method, the heating temperature for removal by heating is not particularly limited provided that the O/W type emulsion can be maintained; however, the temperature can be in the range of 0° C. to 80° C.

In the nanoprecipitation method, the heating temperature for removal by heating is not particularly limited provided that high-order aggregation, decreasing the yield of particles, can be prevented; however, the temperature can be in the range of 0° C. to 80° C.

However, the removal by distillation is not limited to the above techniques in the range allowing the achievement of the object of the present invention.

(Purification of Particle Dispersion)

The purification of the produced particle dispersion can be carried out by any heretofore known method. Examples thereof can include a size-exclusion column chromatography method, an ultrafiltration method, a dialysis method and a centrifuge separation method.

However, the purification method is not limited to the above techniques in the range allowing the achievement of the object of the present invention.

(Particle)

The particle according to each of the present embodiments has any shape provided that the particle is a particle having the above hydrophobic metal phthalocyanine dye, and may be spherical, elliptical, planar, one-dimensional string-like or the like. The size of the particle (particle diameter) according to each of the present embodiments is not particularly limited; however, the particle diameter can be 1 nm to 200 nm (both inclusive).

(Contrast Agent)

The contrast agent according to the present embodiment has the above particles according to the present embodiment and a dispersion medium. The dispersion medium is a liquid substance; examples thereof include saline, distilled water for injection and phosphate buffered saline (hereinafter sometimes abbreviated as PBS). The contrast agent according to the present embodiment may have pharmacologically acceptable additives as needed.

The contrast agent according to the present embodiment may be used by dispersing the above particles in the dispersion medium in advance or by making the particles according to each of the present embodiments and the dispersion medium into a kit and dispersing the particles in the dispersion medium before administration in vivo.

The particle according to each of the present embodiments contains a large amount of the hydrophobic dye therein because the hydrophobic dye is less easily leaked. Because a larger amount of the dye contained increases the amount of light absorption, the particle according to each of the present embodiments is suitable for photoacoustic imaging applications as will hereinafter be described. When a large amount of the hydrophobic dye as to produce concentration quenching is contained, the particle according to each of the present embodiments is more suitable for photoacoustic imaging applications.

(Additive)

The contrast agent according to the present embodiment may contain additives used in lyophilization. Examples of the additive include glucose, lactose, mannitol, polyethylene glycol, glycine, sodium chloride, and sodium hydrogenphosphate. These additives may be used singly or in a combination of a plurality thereof.

(Photoacoustic Imaging Method)

The contrast agent according to the present embodiment can be used for a photoacoustic imaging method. As used herein, the photoacoustic imaging is a concept including photoacoustic tomography (tomographic technique). The photoacoustic imaging method using the contrast agent according to the present embodiment is characterized by at least having a step of administering the contrast agent according to the present embodiment to a specimen or a sample obtained from the specimen, a step of irradiating the specimen or the sample obtained from the specimen with pulsed light and a step of measuring a photoacoustic signal of a substance derived from the particle present in the specimen or the sample obtained from the specimen.

An example of the photoacoustic imaging method using the contrast agent according to the present embodiment is as follows. That is, the contrast agent according to each of the present embodiments is administered to a specimen or added to a sample such as an organ obtained from the specimen. The specimen refers to any living organism such as a human, an experimental animal or a pet without particular limitation; examples of the sample in the specimen or obtained from the specimen can include organs, tissues, tissue sections, cells and cell lysates. After administrating or adding the particles, the specimen or the like is irradiated with laser pulsed light in the near-infrared wavelength region.

In the photoacoustic imaging method according to each of the present embodiments, the wavelength of the irradiated light can be selected depending on the laser light source used. In the photoacoustic imaging method according to each of the present embodiments, irradiation with light of a wavelength in the near-infrared light region of 600 nm to 1,300 nm, called “biological window”, which is less affected by the absorption or diffusion of light in vivo, can be conducted in order to efficiently obtain an acoustic signal.

The photoacoustic signal (acoustic wave) from the contrast agent according to the present embodiment is detected using an acoustic wave detector, for example, a piezoelectric transducer and converted into an electrical signal. Based on the electrical signal obtained through the acoustic wave detector, the location, size or optical characteristic value distribution (such as a molar absorbance coefficient) of an absorber in the specimen or the like can be calculated. For example, if the contrast agent is detected at not less than the threshold used as a reference, the particle-derived substance can be assumed to be present in the specimen, or the particle-derived substance can be assumed to be present in the sample obtained from the specimen.

According to the present invention, the leakage of the dye can be suppressed to produce quenching due to dye accumulation to prevent the energy of the pulsed irradiation light from being used for fluorescent emission for conversion into a greater deal of heat energy. Thus, the acoustic signal can be more efficiently obtained.

In the photoacoustic imaging method according to each of the present embodiments, the wavelength of the irradiation light can be selected depending on the laser light source used.

### EXAMPLES

The present invention will be described below in line with Examples in order to further demonstrate features of the present invention. However, the present invention is not intended to be limited to these Examples, and materials, composition conditions, reaction conditions and the like can freely vary in the range where a contrast agent having the same function and effect is obtained.

The techniques performed in Examples will be described.

(Recovery Method)

Centrifugal separation operation was carried out using a high speed refrigerated micro centrifuge (MX-300 from Tomy Seiko Co., Ltd.). Ultracentrifugal separation operation was carried out using a small ultracentrifuge (CS150GXL from Hitachi Koki Co., Ltd.).

(Analytical Method)

Particle diameter measurement was performed using a dynamic light scattering analysis apparatus (ELSZ-2 from Otsuka Electronics Co., Ltd.).

A semiconductor laser was used as a light source to perform the measurement, and a cumulant diameter value was adopted as a particle diameter.

Absorbance measurement was performed using a UV-VIS-NIR measurement apparatus (Lambda Bio 40 from Perkin Elmer Co., Ltd.).

(Photoacoustic Characteristic Evaluation Method)

The measurement of a photoacoustic signal was carried out by irradiating a sample with pulsed laser light, detecting a photoacoustic signal from the sample using a piezoelectric element, amplifying the signal with a high-speed preamplifier and then obtaining the resultant on a digital oscilloscope. The specific conditions are as follows. A titanium-sapphire laser (from Lotis) was used as a light source. Conditions were used of a wavelength of 790 nm, an energy density of 12 mJ/cm², a pulse width of 20 nanoseconds, and a pulse repeat of 10 Hz. Model V303 (from Panametrics-NDT) was used as an ultrasonic transducer. Conditions therefor were a central band of 1 MHz, an element size of φ0.5, a measurement distance of 25 mm (Non-focus) and an amplifier of +30 dB (ultrasonic preamplifier Model 5682 from Olympus Corporation). A polystyrene cuvette was used as a measurement container, and the light path length and the sample volume thereof were 0.1 cm and about 200 μl, respectively. DPO4104 (from Tektronix, Inc.) was used as a measuring device, and measurement was performed under the conditions of: trigger: detection of photoacoustic light with a photodiode; and data acquisition: 128 times (128 pulses) on average.

(Method for Calculating Molar Absorbance Coefficient per Particle)

The weight concentration of a solid component in a particle dispersion was calculated by lyophilizing the dispersion . . . (A)

Assuming that the density of each constitutional material was 1 (g/cm³), the weight per particle was calculated from the particle diameter of each particle . . . (B)

The weight concentration determined in (A) was divided by the weight per particle determined in (B) to calculate the particle concentration in the particle dispersion . . . (C)

From the results of the absorbance measurement and (C), the molar absorbance coefficient per particle was calculated.

(Method for Evaluating Hydrophobicity of Hydrophobic Metal Phthalocyanine Dye)

To compare the hydrophobicities of hydrophobic metal phthalocyanine dyes, evaluation was carried out using a thin-layer liquid chromatography (hereinafter sometimes abbreviated as TLC) method.

A TLC glass plate, RP-18 (from Merck & Co., Inc.), was used as a development plate, and a methanol solution containing 1% by weight of lithium chloride was used as a development solvent.

According to an established method, a dye solution was spotted on the starting line and the relative migration distance (hereinafter sometimes abbreviated as Rf value) was calculated based on the following equation. Rf value=the distance from the starting line to the center of the spot of the component/the distance from the starting line to the top of the solvent

(Method for Calculating Molar Absorbance Coefficient Per Particle in Terms of 100 nm and Photoacoustic Signal Per Particle in Terms of 100 nm)

The molar absorbance coefficient per particle and the photoacoustic signal per particle were calculated using the respective actual particle diameter data. Then, the respective values were calculated, assuming that the values were proportional to volume ratios when the 100 nm particles were present in the same composition.

### Example 1

### Experimental Example A1

Zinc 2,9,16,23-tetra-tert-butyl-29H,31H-phthalocyanine (8.8 mg) as a dye was dissolved in 16.0 mL of chloroform.

Tween 20 (from Wako Pure Chemical Industries Ltd.) (1,800 mg) as a surfactant was added to 200 mL of ultrapure water to prepare a surfactant aqueous dispersion.

An organic solvent dispersion was added dropwise while stirring the surfactant aqueous dispersion to prepare a preparatory solution for emulsion.

The preparatory solution for emulsion was subjected to ultrasonic irradiation at an intensity scale point of 10 for 1 minute and 30 seconds using an ultrasonic dispersion apparatus (UD-200 from Tomy Seiko Co., Ltd.) to prepare an emulsion.

To remove chloroform in the emulsion, heat-stirring was performed at 40° C. for 4 hours to prepare a particle dispersion.

The recovered particle dispersion was filtered with a filter having a pore diameter of 0.20 micrometer to provide particles (A-1).

### Experimental Example A2

Synthesis was performed in the same way as in Experimental Example A1 except for changing the dye to copper (II) 2,9,16,23-tetra-tert-butyl-29H,31H-phthalocyanine to provide particles (A-2).

### Experimental Example A3

Synthesis was performed in the same way as in Example 1 except for changing the dye to cobalt 2,9,16,23-tetra-tert-butyl-29H,31H-phthalocyanine to provide particles (A-3).

### Experimental Example A4

Synthesis was performed in the same way as in Example 1 except for changing the dye to tert-butyl silicon-[bis trimethylsiloxy]-phthalocyanine to provide particles (A-4).

### Comparative Example B1

Zinc 2,9,16,23-tetra-tert-butyl-29H,31H-phthalocyanine (11.5 mg) as a dye was dissolved in 20.9 mL of chloroform to prepare a dye solution.

The dye solution (0.8 mL) was diluted with 0.8 mL of chloroform to prepare a solution having a dye concentration of 0.28 mg/mL, which was subsequently used.

PLGA (from Wako Pure Chemical Industries Ltd.) (5 mg) as a matrix material was added, followed by preparing an organic solvent dispersion.

Tween 20 (from Wako Pure Chemical Industries Ltd.) (180 mg) as a surfactant was added to 20 mL of ultrapure water to prepare a surfactant aqueous dispersion.

An organic solvent dispersion was added dropwise while stirring the surfactant aqueous dispersion to prepare a preparatory solution for emulsion.

The preparatory solution for emulsion was subjected to ultrasonic irradiation at an intensity scale point of 4 for 1 minute and 30 seconds using an ultrasonic dispersion apparatus (UD-200 from Tomy Seiko Co., Ltd.) to prepare an emulsion.

To remove chloroform in the emulsion, heat-stirring was performed at 40° C. for 2 hours to prepare a particle dispersion.

The resultant particle dispersion was centrifuged at 4° C. and 20,000 G for 45 minutes for recovery.

The recovered particles were washed using ultrapure water. Thereafter, centrifugal separation was carried out at 4° C. and 20,000 G for 45 minutes for recovery.

The recovered particle dispersion was filtered with a filter having a pore diameter of 0.20 micrometer to provide particles (B-1).

### Experimental Example B2

Synthesis was performed in the same way as in Comparative Example B1 except for changing the dye concentration to 0.55 mg/ml to provide particles (B-2).

### Experimental Example B3

Synthesis was performed in the same way as in Comparative Example B1 except for changing the dye concentration to 2.75 mg/mL and not using the matrix material to provide particles (B-3).

Table 1 shows particle diameters and photoacoustic signal intensities (wavelength: 750 nm) per unit dye for the above-obtained particles (A-1) to (A-4). The photoacoustic signal intensities per unit dye were compared.

The results (Rf values) of evaluating the hydrophobicity of the hydrophobic dyes used are also shown.

TABLE 1

| | | | | Photoacoustic |
|---|---|---|---|---|
| | | | Particle | Signal |
| | Hydrophobic metal | Rf | Diameter | Per Unit Dye |
| Particle | phthalocyanine | Value | (nm) | (V J⁻¹ M⁻¹) |
| (A-1) | Zinc 2,9,16,23-tetra-tert- | 0.10 | 15 | 1.0 × 10⁶ |
| | butyl-29H,31H- | | | |
| | phthalocyanine | | | |
| (A-2) | Copper (II) 2,9,16,23-tetra- | 0.06 | 38 | 7.8 × 10⁵ |
| | tert-butyl-29H,31H- | | | |
| | phthalocyanine | | | |
| (A-3) | Cobalt 2,9,16,23-tetra-tert- | 0.08 | 25 | 4.8 × 10⁵ |
| | butyl-29H,31H- | | | |
| | phthalocyanine | | | |
| (A-4) | Tert-butyl silicon-[bis | 0.12 | 28 | 5.2 × 10⁵ |
| | trimethylsiloxy]- | | | |
| | phthalocyanine | | | |

As shown in Table 1, the particles (A-1) to (A-4) prepared in this Example had a high photoacoustic signal intensity (wavelength: 750 nm) per unit dye.

Thus, the particles according to this Example are suitable as contrast media for photoacoustic imaging.

Table 2 shows particle diameters, dye weight percentages in particles, molar absorbance coefficients (wavelength: 750 nm) per particle and photoacoustic signal intensities (wavelength: 750 nm) per particle for the above-obtained particles (B-1) to (B-3). The molar absorbance coefficients per particle and the photoacoustic signal intensities per particle were compared, assuming that the particle diameter was 100 nm.

TABLE 2

| | | Particle | Weight Percentage | Molar Absorbance Coefficient | Photoacoustic Signal Per |
|---|---|---|---|---|---|
| | Hydrophobic metal | Diameter | of Dye in | Per Particle in Terms of | Particle in Terms of |
| Particle | phthalocyanine | (nm) | Particle (%) | 100 nm (M⁻¹ cm⁻¹) | 100 nm (V J⁻¹ M⁻¹) |
| (B-1) | Zinc 2,9,16,23-tetra-tert- | 119 | 1 | 2.4 × 10⁹ | 5.1 × 10⁹ |
| | butyl-29H,31H-phthalocyanine | | | | |
| (B-2) | Zinc 2,9,16,23-tetra-tert- | 108 | 6 | 4.0 × 10⁹ | 1.7 × 10¹⁰ |
| | butyl-29H,31H-phthalocyanine | | | | |
| (B-3) | Zinc 2,9,16,23-tetra-tert- | 105 | 90 | 5.3 × 10⁹ | 3.3 × 10¹⁰ |
| | butyl-29H,31H-phthalocyanine | | | | |

As shown in Table 2, the weight percentage of the dye (hydrophobic metal phthalocyanine) in a particle being 6% or more provided absorption of light at a wavelength of 790 nm, a high molar absorbance coefficient and a high photoacoustic signal. Thus, the contrast media for photoacoustic imaging according to this Example were shown to have absorption in a wavelength region suitable for a photoacoustic imaging method.

As shown in Table 2, the particles (B-2) and (B-3) prepared in this Example were high in the molar absorbance coefficient per unit dye (wavelength: 750 nm) and the photoacoustic signal intensity per unit dye (wavelength: 750 nm) as compared to the particles (B-1) prepared in Comparative Example 1.

Thus, the particles according to this Example are suitable as contrast media for photoacoustic imaging.

### Example 2

### Blood Concentration Quantification Experiment Using Particle

Female outbred BALB/c Slc-nu/nu mice (6 weeks old at purchase) (Japan SLC, Inc.) were used. For 1 week before administration, using a standard diet and a standard bed, the mice were acclimated in an environment allowing the mice to take diet and drink water ad libitum. A particle solution (0.2 mL) was intravenously injected into each acclimated mouse.

The mice receiving administration of the particle solution were not visually found to have any problem after administration; thus, all of the injections were determined to be well tolerated.

Blood was collected 1 and 24 hours after administration. Each collected blood was transferred to a plastic tube, and a 1% Triton-X100 aqueous solution was added at a volume of 4.5 times that of the blood. Then, a volume of 4.5 times that of the blood was added to prepare a blood lysate. Using IVIS (R) Imaging System 200 Series (XENOGEN), the fluorescence intensity of the blood lysate was measured in a state where the lysate is in the plastic tube.

A known concentration of the particle solution was diluted into various concentrations with the 1% Triton-X100 aqueous solution. The diluted particle solutions were each mixed with the same volume of the blood collected from the mouse receiving no administration. Then, the 1% Triton-X100 aqueous solution was added so that the solution combined with the above diluted particle solution had a volume of 4.5 times that of the blood. Subsequently, tetrahydrofuran was added at a volume of 4.5 times that of the blood to prepare blood particle solutions for calibration curve. Fluorescence intensity was measured as with the collected blood samples to prepare a calibration curve.

Then, the fluorescence intensity of the blood lysate and the prepared calibration curve were used to calculate the blood concentration thereof.

The calculated blood concentration was divided by the total amount of administration to calculate the percentage (% ID) of abundance in the blood per administration amount.

Table 3 shows the results of performing the blood concentration quantification experiment using the particles (A-1) and (A-4) obtained above.

The particles (A-1) and (A-4) prepared in Example had a high percentage of abundance in the blood per administration amount.

Thus, the particles probably have a high capability of accumulation in tumor when administered in vivo.

TABLE 3

| | | Percentage of Abundance in Blood per |
|---|---|---|
| | Hydrophobic metal | Administration Amount after 24 Hours |
| Particle | phthalocyanine | (% ID) |
| A-1 | Zinc 2,9,16,23-tetra- | 27 |
| | tert-butyl-29H,31H- | |
| | phthalocyanine | |
| A-4 | Tert-butyl silicon-[bis | 8 |
| | trimethylsiloxy]- | |
| | phthalocyanine | |

### Example 3

### Experiment for Confirming Capability of Accumulation in Tumor Using Particle

Female outbred BALB/c Slc-nu/nu mice (6 weeks old at purchase) (Japan SLC, Inc.) were used. For 1 week before causing the mice to carry cancer, using a standard diet and a standard bed, the mice were acclimated in an environment allowing the mice to take diet and drink water ad libitum. Colon 26 (mouse colon cancer cells) was subcutaneously injected into the mice. All of the tumor cells were fixed by the time of experiment and the body weight of the mice was 17 to 22 g. A contrast agent (100 μL (13 nmol as a dye)) for photoacoustic imaging was intravenously injected into the tail of the cancer-bearing mice.

Then, the mice receiving administration of the contrast agent for photoacoustic imaging were euthanized 24 hours after the administration, followed by removing the colon 26 tumor tissues. The tumor tissues were each transferred to a plastic tube, and a 1% Triton-X100 aqueous solution was added at a weight of 1.25 times that of each tumor tissue, followed by homogenization. Subsequently, tetrahydrofuran (THF) was added at a weight of 20.25 times that of each tumor tissue. The fluorescence intensity of the homogenized solution was measured using Odyssey (R) CLx Infrared Imaging System to quantify the amount of the dye in each tumor tissue.

Table 4 shows the results of measuring the amounts of tumor accumulation of the above-obtained contrast agent (A-1) for photoacoustic imaging and the hydrophilized ZnPc described in Non-Patent Literature 1 as a comparative example.

TABLE 4

| | | Percentage of Abundance in |
|---|---|---|
| | | Tumor per Administration |
| | | Amount after 24 Hours |
| Sample | Dye | (% ID/g) |
| Particle (A-1) | Zinc 2,9,16,23-tetra- | 14 |
| | tert-butyl-29H,31H- | |
| | phthalocyanine | |
| Dye Alone | Hydrophilized ZnPc | 3 |
| (Comparative | | |
| Example) | | |

Thus, the contrast agent for photoacoustic imaging according to this Example has a good capability of accumulation in tumor compared to the hydrophilized ZnPc and is suitable as a contrast agent having a high tumor visualization capability.

### Example 4

### Experimental Example D3

Synthesis was performed in the same way as in Comparative Example B1 except for changing the dye concentration to 2.20 mg/ml and using 20 mg PLGA (from Wako Pure Chemical Industries Ltd.) as a matrix material to provide particles (D-3).

### Experimental Example D4

Synthesis was performed in the same way as in Comparative Example B1 except for changing the dye concentration to 2.20 mg/ml and using 10 mg PLGA (from Wako Pure Chemical Industries Ltd.) as a matrix material to provide particles (D-4).

### Experimental Example D5

Synthesis was performed in the same way as in Comparative Example B1 except for changing the dye concentration to 2.20 mg/ml and using 5 mg PLGA (from Wako Pure Chemical Industries Ltd.) as a matrix material to provide particles (D-5).

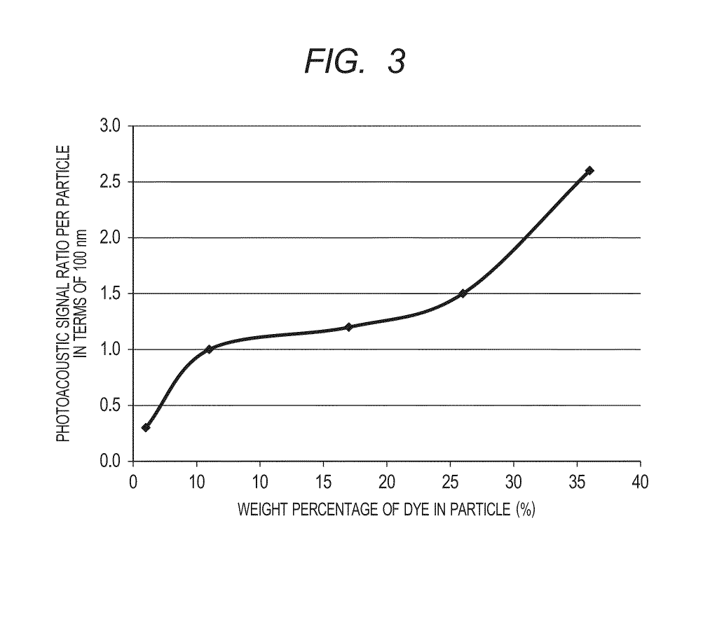

Table 5 shows dye weight percentages and photoacoustic signal (wavelength: 750 nm) per particle indicated as the relative value based on the signal of (B-2) in above-obtained particles (B-1), (B-2), (D-3) to (D-5). The photoacoustic signal intensities per particle were compared, assuming that the particle diameter was 100 nm. The above result is also shown as a graph in FIG. 3. As result, particles of which weight percentage of dye in particle is 6% or more showed high photoacoustic signal. Therefore, particles of which weight percentage of dye in particle is 6% or more is preferable as contrast agent for photoacoustic imaging.

TABLE 5

| | Weight | Photoacoustic Signal Per |
|---|---|---|
| | Percentage | Particle in Terms of 100 |
| | of Dye in | nm (Shown as the relative |
| Particle | Particle (%) | value to B-2 signal) |
| (B-1) | 1 | 0.3 |
| (B-2) | 6 | 1.0 |
| (D-3) | 17 | 1.2 |
| (D-4) | 26 | 1.5 |
| (D-5) | 36 | 2.6 |

The present invention can provide a particle having absorption in a wavelength region suitable for a photoacoustic imaging method and having a high molar absorbance coefficient per particle by increasing the weight percentage of a hydrophobic metal phthalocyanine in the particle.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-158052, filed Jul. 30, 2013, which is hereby incorporated by reference herein in its entirety.

What is claimed is:

1. A contrast agent for photoacoustic imaging, the contrast agent being a particle comprising a hydrophobic metal phthalocyanine and a surfactant, wherein the weight percentage of the hydrophobic metal phthalocyanine in the particle is 6% or more.

2. The contrast agent for photoacoustic imaging according to claim 1, wherein the hydrophobic metal phthalocyanine is represented by general formula (1): 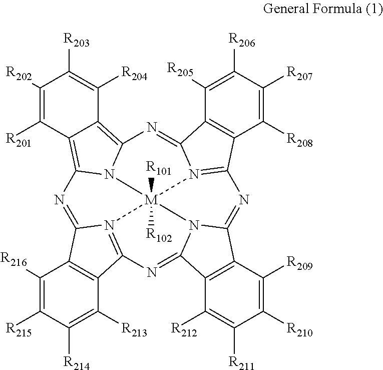
wherein R₂₀₁ to R₂₁₆ may be identical or different and each represent a hydrogen atom, a halogen atom, an acetoxy group, an amino group, a nitro group, a cyano group or an alkyl group having 1 to 18 carbons or an aromatic group, wherein the aromatic group is one unsubstituted or substituted with one or more functional groups selected from the group consisting of halogen atoms, an acetoxy group, an amino group, a nitro group, a cyano group, and alkyl groups each having 1 to 18 carbons;
M represents a Zn, Cu, Co or Si element;
R₁₀₁ and R₁₀₂ may be identical or different, may be absent depending on the element of M, or are each represented by the structure shown below:
—OH, —OR₁₁, —OCOR₁₂, —OSi(—R₁₃)(—R₁₄)(—R₁₅), a halogen atom, an acetoxy group, an amino group, a nitro group, a cyano group or an alkyl group having 1 to 18 carbons or an aromatic group, wherein the aromatic group is one unsubstituted or substituted with one or more functional groups selected from the group consisting of halogen atoms, an acetoxy group, an amino group, a nitro group, a cyano group, and alkyl groups each having 1 to 18 carbons,
wherein R₁₁ to R₁₅ are each selected from the group consisting of halogen atoms, an acetoxy group, an amino group, a nitro group, a cyano group, and alkyl groups each having 1 to 18 carbons, wherein R₁₃, R₁₄ and R₁₅ may be identical or different.

3. The contrast agent for photoacoustic imaging according to claim 1, wherein the hydrophobic metal phthalocyanine is zinc 2,9,16,23-tetra-tert-butyl-29H,31H-phthalocyanine.

4. The contrast agent for photoacoustic imaging according to claim 1, wherein the surfactant is a non-ionic surfactant.

5. The contrast agent for photoacoustic imaging according to claim 1, wherein the surfactant is a polyoxyethylene sorbitan fatty acid ester.

6. The contrast agent for photoacoustic imaging according to claim 1, wherein the surfactant is Tween 20 or Tween 80.

7. The contrast agent for photoacoustic imaging according to claim 1, wherein the particle further comprises a matrix material.

8. The contrast agent for photoacoustic imaging according to claim 7, wherein the matrix material is a hydrophobic polymer.

9. The contrast agent for photoacoustic imaging according to claim 1, wherein the particle has a particle diameter of 200 nm or less.

10. A contrast agent for photoacoustic imaging, comprising the particle according to claim 1 and a dispersion medium.

11. The contrast agent for photoacoustic imaging according to claim 10, further comprising an additive.
